(19) United States
(12) Plant Patent Application Publication
(10) Pub. No.: US 2006/0070155 P1
(43) Pub. Date: Mar. 30, 2006

(54) VIOLA PLANT NAMED 'SUNVIOBUHO'

(50) VIOLA CORNUTA; VIOLA

(76) Inventors: Kiyoshi Miyazaki, Hikone (JP); Naoto Takamura, Rittou (JP)

Correspondence Address: Ronald L. Grudziecki;BURNS, DOANE, SWECKER & MATHIS, L.L.P, P.O. Box 1404, Alexandria, VA, 22313-1404, US

(21) Appl. No.: 10/949,334

(22) Filed: Sep. 27, 2004

(51) Int. Cl. A01H 5/00

(52) U.S. Cl. PLT/323

(57) ABSTRACT

Disclosed herein is a new and distinct variety of Viola cornuta, named ‘Sunviobuho’, which is a vegetatively propagated variety. This new variety has a spreading growth habit with long prostrate stems. This Viola plant has many branches, and great profusion of blooms with the entire plant remaining in bloom for a considerable period of time. It displays dense green foliage, and single, small-sized flowers on long peduncles that are violet-blue and white in coloration. The plant exhibits good resistance to cold, diseases and pests.

### BOTANICAL CLASSIFICATION/CULTIVAR DESIGNATION

Viola cornuta/Viola Plant

### BACKGROUND OF THE VARIETY

The present invention relates to a new and distinct variety of Viola cornuta named ‘Sunviobuho’ originated from the crossing a Viola hybrid variety called ‘0V-41-9’ as the female parent and ‘0V-80’ as the male parent.

In April 2000, crossing of ‘0V-41-9’ as the female parent and ‘0V-80’ as the pollen parent was conducted at Yokaichi-shi, Shiga, Japan. In May 2000, some seeds were obtained from that crossing. In August 2000, the seeds were sown and 20 seedlings were obtained. Three of the seeding were selected in view of their spreading growth habit, small flower size and blue and white petal color. Those seedlings were propagated by cutting and grown in pots, and then a trial was carried out from October 2001, at Yokaichi-shi, Shiga, Japan. The botanical characteristics of that plant were then examined, using similar varieties, ‘Violetto Nive’ and ‘Alpine Summer’, for comparison. As a result, one seedling was selected and it was concluded that this Viola plant is distinguishable from any other variety, whose existence is known to us, and is uniform and stable in its characteristics. The new variety of Viola plant was named ‘Sunviobuho’.

The female parent ‘0V-41-9’ (not patented in the U.S.) is our breeding line grown at Yokaichi-shi, Shiga, Japan, having spreading growth habit with prostrate stems. It has smaller size flowers than the new variety ‘Sunviobuho’ and bi-colored flowers consist of yellow and white color.

The pollen parent ‘0V-80’ (not patented in the U.S.) is our breeding line grown at Yokaichi-shi, Shiga, Japan, having spreading growth habit with prostrate stems. It has small size flowers than the new variety ‘Sunviobuho’, the petals having light blue color.

In the following description, the color information is in accordance with The R.H.S. Colour Chart of The Royal Horticultural Society, London, England.

### SUMMARY OF THE INVENTION

This new variety is unlike any Viola cornuta commercially available as evidenced by the following unique combinations of characteristics.

1. Spreading growth habit having long prostrate stems and many branches.

2. Great profusion of blooms, the whole plant remaining in bloom for a considerable period of time.

3. Single, small size flowers having blue and white multi-colored petals, on the long peduncles.

The new variety ‘Sunviobuho’ differs from the similar variety ‘Violetto Nive’ (not patented in the U.S.) in the following points.

1. The peduncle length of ‘Sunviobuho’ is longer than that of ‘Violetto Nive’.

2. The flower of ‘Sunviobuho’ is smaller than that of ‘Violetto Nive’.

3. The flower of ‘Sunviobuho’ and ‘Violetto Nive’ differs in petal color. ‘Sunviobuho’ is bi-colored, the upper petal is blue, lateral and lower petals are white. That of ‘Violetto Nive’ is White.

4. Flowering time of ‘Sunviobuho’ is earlier than that of ‘Violetto Nive’.

The new variety ‘Sunviobuho’ differs from the similar variety ‘Alpine Summer’ (not patented in the U.S.) in the following points.

1. The peduncle length of ‘Sunviobuho’ is longer than that of ‘Alpine Summer’.

2. The flower of ‘Sunviobuho’ is larger than that of ‘Alpine Summer’.

3. The flower of ‘Sunviobuho’ and ‘Violetto Nive’ differs in petal color. ‘Sunviobuho’ is bi-colored, the upper petal is blue, lateral and lower petals are white. That of ‘Alpine Summer’ is bi-colored, the upper petal is violet, lateral and lower petals are yellow with dark purple vein.

4. Flowering time of ‘Sunviobuho’ is earlier than that of ‘Alpine Summer’.

The new variety of Viola plant ‘Sunviobuho’ was asexually reproduced by the use of cuttings at Yokaichi-shi, Shiga, Japan, and the homogeneity and stability thereof were confirmed.

The instant plant retains its distinctive characteristics and reproduces true to type in successive generations.

### BRIEF DESCRIPTION OF THE PHOTOGRAPHS

The accompanying photographs show, as nearly true as it is reasonably possible to make the same in color illustrations of this character, typical specimens of the new cultivar. The plants had been reproduced by the use of cuttings and were photographed May 2002 while growing outdoors in 24 cm pots at an age of approximately 7 months at Yokaichi-shi, Shiga, Japan.

FIG. 1 illustrates an entire plant of the new variety while growing in a pot.

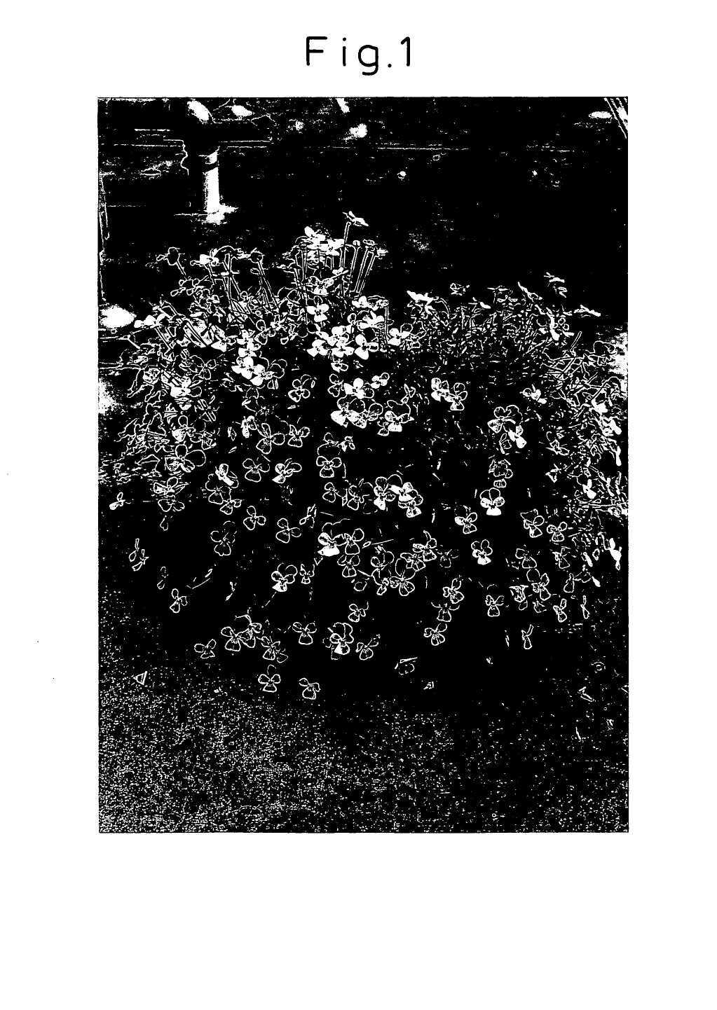

FIG. 2 illustrates a close view of the foliage and blossoms of the new variety.

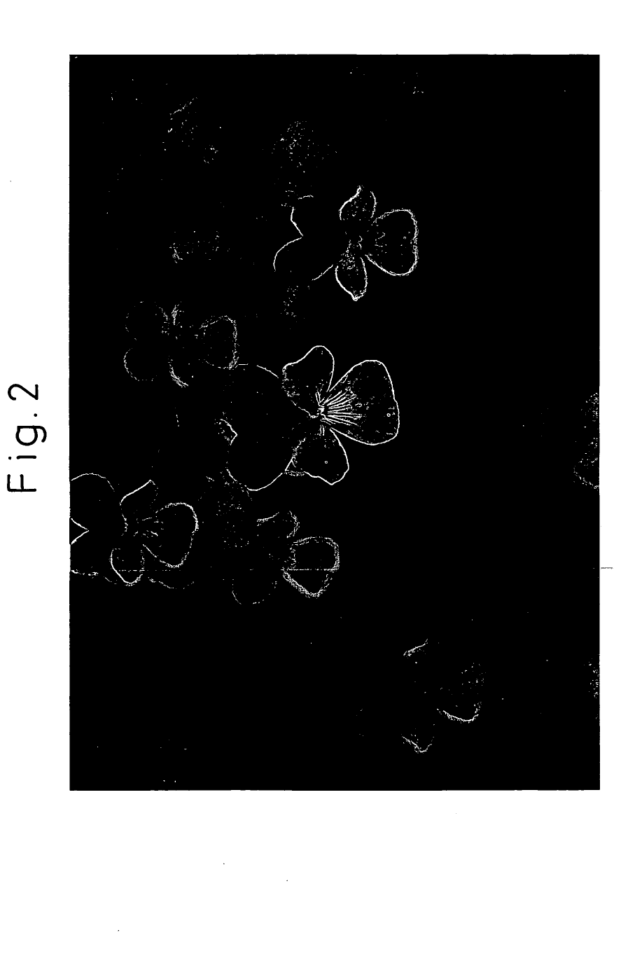

### DESCRIPTION OF THE NEW VARIETY

The botanical characteristics of the new and distinct variety of Viola cornuta named ‘Sunviobuho’ are as follows were observed during May 2002 at Yokaichi-shi, Shiga, Japan, at an age of approximately 7 months.
Plant:
   Stemmed or stemless.—Stemmed.
   Growth habit.—Spreading.
   Height.—Approximately 14 cm.
   Spreading area.—Approximately 60 cm.
Stem:
   Length.—Approximately 38 cm.
   Color.—R.H.S.144B (Green group).
   Branches.—Many.
   Pubescence.—Present.
   Internode length.—Approximately 1.5 cm.
Leaf:
   Phyllotaxis.—Alternate.
   Overall shape.—Lanceolate.
   Apex shape.—Obtuse.
   Base shape.—Obtuse.
   Margin.—Crenate.
   Length.—Approximately 3.0 cm.
   Width.—Approximately 1.4 cm.
   Color of upper surface.—R.H.S.137A (Green group).
   Color of under surface.—R.H.S.137C (Green group).
   Leaf texture.—Matte and smooth.
   Venation.—Reticulate venation, not apparent except center venation.
   Petiole length.—Approximately 2.7 cm.
   Petiole diameter.—Approximately 0.5 mm.
   Stipule.—Approximately 2.5 cm in length, 1.3 cm in width. Pinnately parted. Shape is narrowly ovate. Color of upper surface. — R.H.S.137A (Green group). Color of under surface. — R.H.S.137C (Green group).
Flower:
   Inflorescence.—Flower solitary, axillary.
   Flower form.—Single, 5 petals, 2 upper petals, 2 lateral petals and a spurred lower petal.
   Longitudinal diameter.—Approximately 3.3 cm.
   Horizontal diameter.—Approximately 2.5 cm.
   Petal margin.—Entire, flat.
   Flower color.—Multi-colored. Upper petal: R.H.S.91A (Violet-blue group). Lateral petal: R.H.S.155A (White up). Lower petal: R.H.S.155A (White group).
   Petal size.—(Length×width) Upper petal: Approximately 1.8 cm×1.4 cm. Lateral petal: Approximately 1.2 cm×0.8 cm. Lower petal: Approximately 1.3 cm×1.5 cm.
   Petal shape.—Upper petal: Rounded. Lateral petal: Elliptical. Lower petal: Cordate, spurred.
   Eye color.—R.H.S.17B (Yellow-orange group) on the lower petal.
   Eye size.—Length: Approximately 2.2 mm. Width: Approximately 2.4 mm.
   Spur length.—Approximately 4.5 mm.
   Spur diameter.—Approximately 1.0 mm.
   Spur color.—R.H.S.116B (Blue-green group).
   Sepal.—5 sepals, unequal, approximately 0.6 cm-1.2 cm, extended into short appendage.
   Number of flowers.—November to March: Approximately 20 flowers per a plant. March to June: Approximately 100-120 flowers per a plant.
   Peduncle length.—Approximately 14.5 cm.
   Peduncle thickness.—Approximately 0.8 mm.
   Peduncle color.—R.H.S.137C (Green group).
Reproductive organs:
   Stamen.—Five stamens joined around the ovary. Two lower stamens spurred.
   Stamen length.—Approximately 2.0 mm.
   Anther color.—R.H.S. 9D (Yellow group). Tip: R.H.S. 166C (Greyed-orange group).
   Pollen color.—R.H.S. 9D (Yellow group).
   Pistil length.—Approximately 4.5 mm.
   Stigma shape.—Ellipsoidal.
   Stigma length.—Approximately 1.0 mm.
   Stigma color.—R.H.S. 144B (Yellow-green group).
   Style length.—Approximately 1.0 mm.
   Style color.—R.H.S. 144D(Yellow-green group).
   Ovary color.—R.H.S. 144C(Yellow-green group).
Seeds:
   Seed.—Some seed development has been observed.
   Seeds color.—R.H.S. 165A (Greyed-orange group).
   Size.—Approximately 2.0 mm in length, 1.0 mm in width.
Physical property:
   Blooming period.—Early November to June in Japan, except Hokkaido. A bloom commonly lasts 5-7 days on the plant.
   Fragrance.—Absent.
   Rain tolerance.—Medium.

This variety has survived temperatures of at least −7° C.

The plant grows well at temperature up to 25° C.

The lower and upper temperature for the plant survival has not been evaluated.

No serious damage by white fly, aphids, and fungus and bacterial diseases has been observed.

This new variety of Viola cornuta plant ‘Sunviobuho’ is most suitable for potting, hanging basket and flower bedding.

1. A new and distinct variety of Viola cornuta plant named ‘Sunviobuho’, substantially as herein illustrated and described.
